(19) United States
(12) Patent Application Publication
(10) Pub. No.: US 2006/0004283 A1
(43) Pub. Date: Jan. 5, 2006

(54) Medical image generating system and medical image generating method

(76) Inventors: Naoto Moriyama, Tokyo (JP); Mamoru Umeki, Tokyo (JP)

Correspondence Address: CANTOR COLBURN, LLP, 55 GRIFFIN ROAD SOUTH, BLOOMFIELD, CT, 06002, US

(21) Appl. No.: 11/167,834

(22) Filed: Jun. 27, 2005

(30) Foreign Application Priority Data
Jul. 5, 2004 (JP) 2004-197979
Jul. 5, 2004 (JP) 2004-198104

(51) Int. Cl. A61B 5/05

(52) U.S. Cl. 600/416; 600/414

(57) ABSTRACT

A medical image generating system for generating a medical image including a subject image and a marker image indicating information of an image generation by generating an image of a subject and by generating an image of a marker for providing the information of the image generation, includes: a marker image editing section for editing the marker image.

### BACKGROUND OF THE INVENTION

1. Field of the Invention

The present invention relates to a medical image generating system and a medical image generating method for generating a medical image of a subject.

2. Description of Related Art

An image generated by using a radiation has been widely used as a medical image for a diagnosis. For example, it is disclosed that an art for outputting an image by setting a photostimulable phosphor sheet on which a radiation image has been recorded by radiography at a predetermined position of a reading unit, by making the reading unit read the radiation image recorded on the photostimulable phosphor sheet, by inputting the obtained image data to a processing unit, by applying an image processing on the image data based on a separately-inputted radiography condition and by structuring an output image based on output form information which is preliminarily stored in a database according to the radiography condition (for example, see JP-Tokukai-2002-158862A).

By the way, upon a diagnosis of a mamma, a diagnosis is performed by an interpretation comparing related images, between left and right or different directions. Therefore, in general, when a mamma image generation is performed, with respect to one subject, a plurality of times of image generations with different image generation conditions (body part and direction) are performed such as a left mamma in up and down directions (CC(Cranio-Caudal)-L(left)), a left mamma in an oblique direction (MLO(Medio-Lateral Oblique)-L), a right mamma in up and down directions (CC-R(right)) and a right mamma in an oblique direction (MLO-R).

Therefore, when a mamma image generation is performed by using a portable recording medium such as a portable photostimulable phosphor sheet or the like, an actual image generation condition for the image generation and an image generation condition preliminarily corresponded to image generation order information are required not to be different from each other.

However, in the art disclosed in JP-Tokukai-2002-158862A, an image generation direction is identified based on an image generation condition inputted at the processing unit to the image data. Therefore, even when a difference between the image generation condition for the actual image generation and the image generation condition to be related to the image data occurs, there is a possibility of not noticing the difference.

Accordingly, so far, in order to prevent a mistake on image generation conditions, upon generating a mamma image, an image of a lead marker indicating a distinction of left or right or indicating an image generation direction is generated along with the mamma image generation. Thereby, it is possible to display a marker image indicating information upon the image generation, such as an image generation condition for the actual image generation, a distinction of left or right of the image-generated mamma and the like, on the image, whereby it is preferable.

However, there is a case in which an image generation is performed without a lead marker placed, a case in which an image generation is performed with a wrong lead marker placed, or a case in which an image generation is performed with a wrong recording medium used. In such a case, it is not possible to edit a marker image to be formed on the medical image, and thereby there is a case of storing or outputting a medical image still including the wrong marker image. Further, when an image generation is performed with a wrong maker placed or when a wrong recording medium is used upon the image generation, it is possible to have a situation where the information indicated by the marker image on the medical image is different from image, generation accompanying information such as an image generation condition or the like, which is preliminarily related to the medical image. In such a case, it is not either possible to make a correction.

### SUMMARY OF THE INVENTION

An object of the present invention is to make it possible to edit a marker image on a medical image for diagnosis.

To solve the above problem, in accordance with the first aspect of the present invention, a medical image generating system for generating a medical image includes a subject image and a marker image indicating information of an image generation by generating an image of a subject and by generating an image of a marker for providing the information of the image generation, and the medical image generating system comprises: a marker image editing section for editing the marker image.

According to the first aspect of the present invention, when an image generation is performed without a lead marker placed or with a wrong lead marker, it is possible to edit a marker image included in a medical image and to prevent storing or outputting a medical image including a wrong marker image.

Preferably, the marker image editing section corrects a part of or all of the marker image.

According to this invention, when an image generation is performed with a wrong marker, it is possible to correct a marker image included in a medical image and showing wrong information to be a marker image showing correct information.

Preferably, the marker image editing section deletes a part of or all of the marker image by converting a density of the part of or all of the marker image into a density equal to a density of an area which is outside of an area of the subject image and outside of an area of the marker image, on the medical image.

According to this invention, when an image generation is performed with a wrong marker, it is possible to delete a marker image included in a medical image and showing wrong information.

Preferably, the marker image editing section adds a new marker image on an area which is outside of the subject area of the medical image when the medical image does not include the marker image.

According to this invention, when an image generation is performed without a marker placed, it is possible to add a new marker image on an area which is outside of the subject area of the medical image.

Preferably, the medical image generation system further comprises a storing section for storing data of the medical image on which the marker image has been edited.

According to this invention, it is possible to store data of the medical image including an edited marker image showing correct information.

Preferably, the medical image generation system further comprises a relating section for relating the data of the edited medical image with image generation accompanying information relating to the medical image.

According to this invention, it is possible to relate the data of the medical image including an edited marker image showing correct information with image generation accompanying information.

Preferably, the medical image generation system further comprises: a reading apparatus for performing the image generation by using a cassette comprising a photostimulable phosphor plate and for obtaining the medical image by reading an image recorded in the photostimulable phosphor plate of the cassette; and a control apparatus for applying a predetermined process on the medical image obtained by the reading apparatus, wherein the control apparatus comprises the marker image editing section.

According to this invention, it is possible to edit a marker image included in a medical image in a control apparatus of the medical image generation system that further comprises: a reading apparatus for performing the image generation by using a cassette comprising a photostimulable phosphor plate and for obtaining the medical image by reading an image recorded in the photostimulable phosphor plate of the cassette; and the control apparatus for applying a predetermined process on the medical image obtained by the reading apparatus.

Preferably, the medical image generating system performs the image generation by using a cassette type flat panel detector, the medical image generating system further comprises a control apparatus for applying a predetermined process on the medical image obtained by the flat panel detector, and the control apparatus comprises the marker image editing section.

According to this invention, it is possible to edit a marker image included in a medical image, in a control apparatus of the medical image generating system that performs the image generation by using a cassette type flat panel detector, and further comprises the control apparatus for applying a predetermined process on the medical image obtained by the flat panel detector.

Preferably, the marker image editing section comprises an operator identification information inputting section for inputting operator identification information for identifying an operator, and when the inputted operator identification information is permitted operator identification information, the editing is allowed.

According to this invention, it is possible to prevent anyone other than an allowed operator from editing a marker image needlessly.

In accordance with the second aspect of the present invention, a medical image generating system for generating a medical image including a marker image indicating information of an image generation by generating an image of a subject and by generating an image of a marker, the medical image generating system comprises: a display section for displaying a display screen; a control section for simultaneously displaying the medical image including the generated marker image and image generation accompanying information corresponding to the medical image; and a correcting section for correcting the marker image included in the displayed medical image or for correcting the image generation accompanying information.

According to the second aspect of the present invention, an operator such as an image generation technician (radiologist) can compare a marker image included in a medical image with image generation accompanying information corresponding to the medical image to check whether there is a difference between them, and in case that there is a difference, can correct the marker image or the image generation accompanying information.

Preferably, the medical image generating system performs the image generation by using a cassette comprising a photostimulable phosphor plate, the medical image generating system comprises a reading apparatus for obtaining the medical image by reading an image recorded in the photostimulable phosphor plate of the cassette and an information processing apparatus for applying a predetermined process on the medical image obtained by the reading apparatus, and the information processing apparatus comprises the display section, the control section and the correcting section.

According to this invention, an operator such as an image generation technician can compare a marker image included in a medical image with image generation accompanying information corresponding to the medical image to check whether there is a difference between them, and in case that there is a difference, can correct the marker image included in the displayed medical image or the image generation accompanying information corresponding to the medical image, in the medical image generating system that performs the image generation by using a cassette comprising a photostimulable phosphor plate and comprises a reading apparatus for obtaining the medical image by reading an image recorded in the photostimulable phosphor plate of the cassette and an information processing apparatus for applying a predetermined process on the medical image obtained by the reading apparatus.

Preferably, the medical image generating system performs the image generation by using a cassette type flat panel detector, the medical image generating system comprises an information processing apparatus for applying a predetermined process on the medical image obtained by the flat panel detector, and the information processing apparatus comprises the display section, the control section and the correcting section.

According to this invention, an operator such as an image generation technician can compare a marker image included in a medical image with image generation accompanying information corresponding to the medical image to check whether there is a difference between them, and in case that there is a difference, can correct the marker image included in the displayed medical image or the image generation accompanying information corresponding to the medical image, in the medical image generating system that performs the image generation by using a cassette type flat panel detector, and comprises an information processing apparatus for applying a predetermined process on the medical image obtained by the flat panel detector.

In accordance with the third aspect of the present invention, a medical image generating system for generating a plurality of medical images with respect to one subject,

the plurality of medical images including a marker image indicating information of an image generation, by performing a mamma image generation under a plurality of image generation conditions with respect to the one subject and by generating an image of a mamma and an image of a marker for providing the information of the image generation, the medical image generating system comprises: a display section for displaying a display screen; a control section for simultaneously displaying the plurality of medical images including the marker image generated with respect to the one subject on the display screen of the display section so as to arrange the plurality of medical images in a turn of the image generation; and a correcting section for correcting the marker image included in the plurality of displayed medical images.

According to the third aspect of the present invention, when a mamma image generation is performed with respect to one subject under a plurality of image generation conditions, an operator such as an image generation technician can determine whether the marker image correctly shows information at the time of image generation based on the turn of the image generation and correct a marker image on a medical image.

Preferably, the control section simultaneously displays the plurality of medical images including the marker image generated with respect to the one subject on the display screen of the display section, so as to relate the plurality of medical images with image generation accompanying information corresponding to each of the plurality of medical images and to arrange the plurality of medical images in the turn of the image generation, and

the correcting section corrects the marker image included in the displayed medical images or corrects the image generation accompanying information.

According to this invention, when a mamma image generation is performed with respect to one subject under a plurality of image generation conditions, an operator such as an image generation technician can compare a marker image included in a medical image with image generation accompanying information corresponding to the medical image to check whether there is a difference between them, and in case that there is a difference, can determine whether the marker image correctly shows information at the time of image generation based on the turn of the image generation and correct the marker image on a medical image or the image generation accompanying information corresponding to the medical image.

Preferably, the medical image generating system performs the image generation by using a cassette comprising a photostimulable phosphor plate, the medical image generating system comprises a reading apparatus for obtaining a medical image recorded in the photostimulable phosphor plate of the cassette and an information processing apparatus for applying a predetermined process on the medical image obtained by the reading apparatus, and the information processing apparatus comprises the display section, the control section and the correcting section.

According to this invention, when a mamma image generation is performed with respect to one subject under a plurality of image generation conditions, an operator such as an image generation technician can compare a marker image included in a medical image with image generation accompanying information corresponding to the medical image to check whether there is a difference between them, and in case that there is a difference, can determine whether the marker image correctly shows information at the time of image generation based on the turn of the image generation and correct the marker image on a medical image or the image generation accompanying information corresponding to the medical image, in the medical image generating system that performs the image generation by using a cassette comprising a photostimulable phosphor plate, and comprises a reading apparatus for obtaining a medical image recorded in the photostimulable phosphor plate of the cassette and an information processing apparatus for applying a predetermined process on the medical image obtained by the reading apparatus.

Preferably, the medical image generating system performs the image generation by using a cassette type flat panel detector, the medical image generating system comprises an information processing apparatus for applying a predetermined process on a medical image obtained by the flat panel detector, and the information processing apparatus comprises the display section, the control section and the correcting section.

According to this invention, when a mamma image generation is performed with respect to one subject under a plurality of image generation conditions, an operator such as an image generation technician can compare a marker image included in a medical image with image generation accompanying information corresponding to the medical image to check whether there is a difference between them, and in case that there is a difference, can determine whether the marker image correctly shows information at the time of image generation based on the turn of the image generation and correct the marker image on a medical image or the image generation accompanying information corresponding to the medical image, in the medical image generating system that performs the image generation by using a cassette type flat panel detector and comprises an information processing apparatus for applying a predetermined process on a medical image obtained by the flat panel detector.

In accordance with the fourth aspect of the present invention, there is provided a medical image generating method for generating a plurality of medical images with respect to one subject, the plurality of medical images including a marker image indicating information of an image generation, by performing a mamma image generation under a plurality of image generation conditions with respect to the one subject and by generating an image of a mamma and an image of a marker for providing the information of the image generation, wherein by simultaneously displaying the plurality of medical images including the marker image generated with respect to the one subject on the display screen of the display section in a turn of the image generation, it is allowed to correct the marker image.

According to the fourth aspect of the present invention, an operator such as an image generation technician can determine whether the marker image correctly shows information at the time of image generation based on the turn of the image generation and correct the marker image on a medical image.

Preferably, by simultaneously displaying the plurality of medical images including the marker image generated with respect to the one subject on the display screen of the display section in a turn of the image generation, so as to relate each of the plurality of medical images with image generation accompanying information corresponding to the each of the plurality of medical images, it is allowed to correct the marker image or the image generation accompanying information.

According to this invention, when a mamma image generation is performed with respect to one subject under a plurality of image generation conditions, an operator such as an image generation technician can compare a marker image included in a medical image with image generation accompanying information corresponding to the medical image to check whether there is a difference between them, and in case that there is a difference, can determine whether the marker image correctly shows information at the time of image generation based on the turn of the image generation and correct the marker image on a medical image or the image generation accompanying information corresponding to the medical image.

### BRIEF DESCRIPTION OF THE DRAWINGS

The present invention will become more fully understood from the detailed description given hereinafter and the accompanying drawing given by way of illustration only, and thus are not intended as a definition of the limits of the present invention, and wherein:

FIG. 1 is a view showing a whole structure of a medical image generating system 100 relating to the present invention,

FIG. 2 is a block diagram showing a functional structure of a controller 3 of FIG. 1,

FIG. 3 is a view showing a structure example of a storing unit 35 of FIG. 2,

FIG. 4 is a flowchart illustrating a mamma image generating process executed by a CPU 31 of FIG. 2,

FIG. 5 is a view showing a data storing example of an image generation accompanying information storing unit 352 of FIG. 3,

FIG. 6 is a view showing one example of an image confirmation screen 331,

FIG. 7 is a flowchart illustrating a marker image editing process executed by the CPU 31 of FIG. 2,

FIG. 8 is a view showing one example of an image generation turn display screen 332,

FIG. 9 is a flowchart illustrating an L/R correction process executed by the CPU 31 of FIG. 2, and

FIG. 10 is a view showing a whole structure of a medical image generating system 200 relating to the present invention.

### PREFERRED EMBODIMENTS OF THE INVENTION

Hereinafter, an embodiment will be described with reference to drawings.

First, a structure will be described.

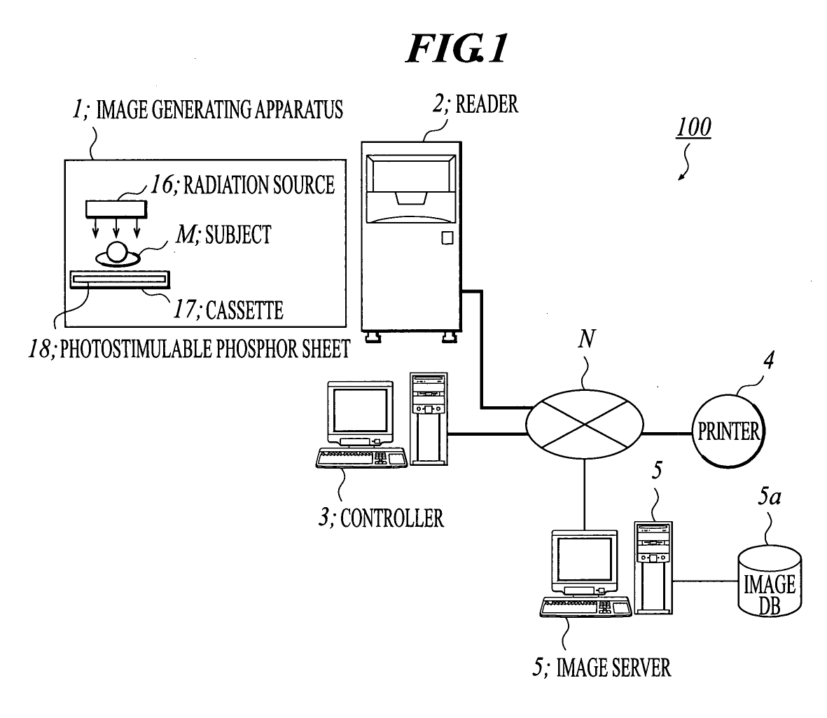

FIG. 1 shows a whole structure of a medical image generating system 100 in the present embodiment. As shown in FIG. 1, the medical image generating system 100 comprises an image generating apparatus 1, a reading apparatus (hereafter, it is referred to as “reader”) 2, a control apparatus (hereafter, it is referred to as “controller”) 3, a printer 4, an image server 5 comprising an image DB 5a, etc. The reader 2, the controller 3, the printer 4 and the image server 5 are connected to each other through a network N so as to make it possible to mutually transmit/receive data. Here, the number of each apparatus is not specifically limited. Further, an external device or a system such as a viewer, an HIS(Hospital Information System)/RIS(Radiology Information System) and the like, may be connected to the network N.

As the network N, various line forms such as a LAN (Local Area Network), a WAN (Wide Area Network), Internet and the like are applicable. Here, as long as permitted to be used in a medical institution such as a hospital, a wireless communication or an infrared ray communication may be used. However, since including important patient information, information to be transmitted/received is preferably encoded. Further, as a communication protocol in the hospital, in general, DICOM (Digital Image and Communications in Medicine) standard is used. For the above-mentioned communication among each apparatus on the network N, DICOM MWM (Modality Worklist Management) or DICOM MPPS (Modality Performed Procedure Step) is used.

The image generation apparatus 1 comprises a radiation source 16 for irradiating a radiation to a subject M for performing an image generation. A cassette 17 is a portable radiation image conversion medium incorporating therein a radiation image conversion plate comprising a photostimulable phosphor sheet 18 for accumulating radiation energy. Radiation dose corresponding to a radiation transmittance distribution of the subject M with respect to the irradiated radiation amount from the radiation source 16 is accumulated in a photostimulable phosphor layer placed in the photostimulable phosphor sheet 18, and radiation image information of the subject M is recorded in the photostimulable phosphor layer.

The reader 2 is an image generating apparatus for generating a medical image by reading the radiation image information of the subject recorded in the attached cassette 17. The reader 2 irradiates an excitation light to the photostimulable phosphor sheet of the attached cassette 17, photoelectrically converts a photostimulated light emitted from the sheet, and A/D converts the obtained image signal, for generating medical image data.

The controller 3 is a control apparatus in the present invention. The controller 3 inputs image generation order information such as patient information, image generation part information, image generation direction information and the like, displays an image based on the medical image data read by the reader 2, performs an image processing on the read medical image data, edits marker image data included in the read medical image data, relates the read medical image data with the image generation accompanying information including the image generation order information, and the like.

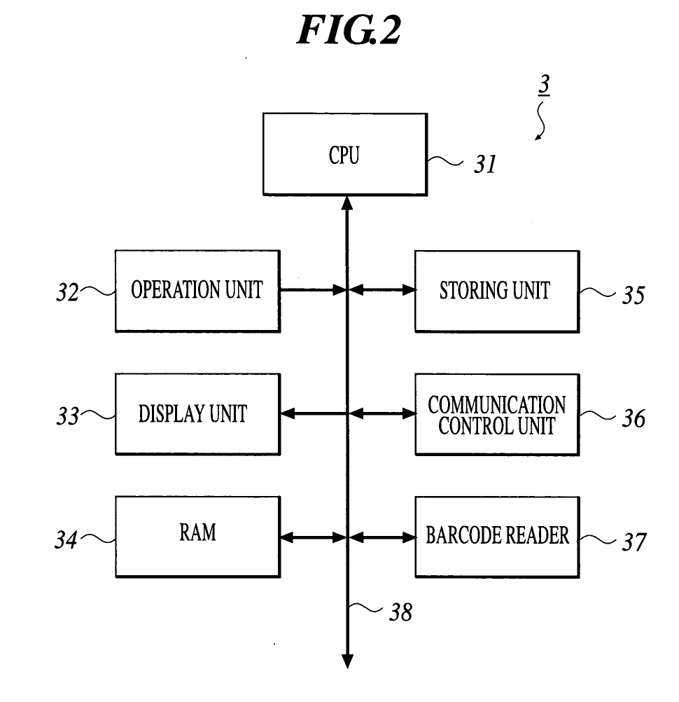

Hereinafter, an internal structure of the controller 3 will be described with reference to FIG. 2.

As shown in FIG. 2, the controller 3 comprises a CPU 31, an operation unit 32, a display unit 33, a RAM 34, a storing unit 35, a communication control unit 36, a barcode reader 37, etc. In the controller 3, each unit is connected by a bus 38.

The CPU 31 reads out a system program stored in a program storing unit 351 of the storing unit 35, develops the read system program into a work area formed in the RAM 34, and controls each unit according to the system program. Further, the CPU 31 reads out various processing programs and various application programs, such as a mamma image generating process program and the like, stored in the program storing unit 351 of the storing unit 35, and executes various processes such as a mamma image generating process which will be described later.

The operation unit 32 comprises a keyboard comprising cursor keys, numeral input keys, various function keys and the like, and a pointing device such as a mouse or the like. The operation unit 32 outputs an instruction signal inputted according to a key operation to the keyboard or a mouse operation to the CPU 31. Further, the operation unit 32 may be provided on a display screen of the display unit 33 as a touch panel. In this case, an inputted instruction signal is outputted to the CPU 31 through the touch panel.

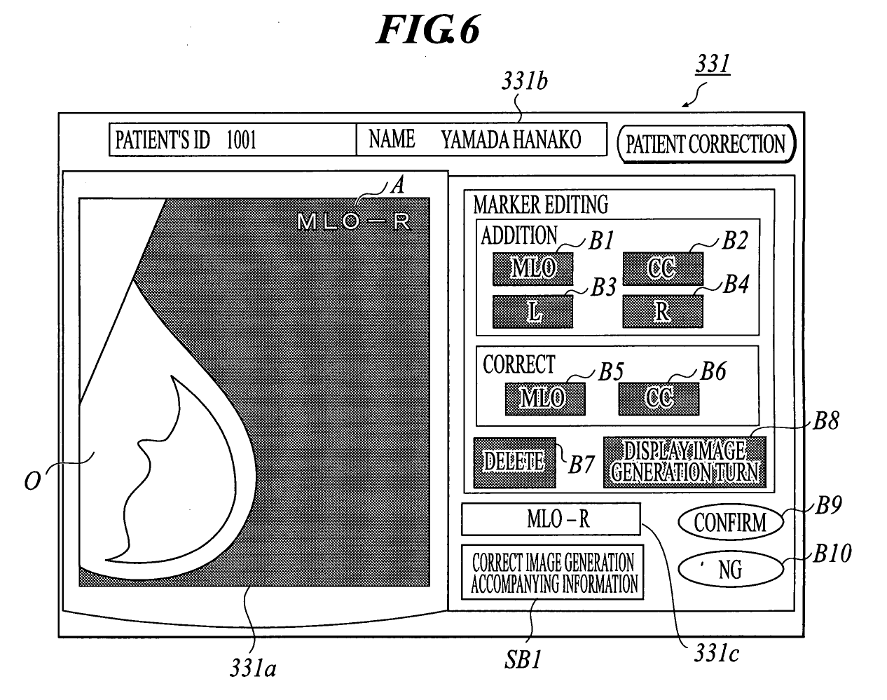

The display unit 33 comprises a monitor such as an LCD (Liquid Crystal Display), a CRT or the like, and shows a display screen such as an image confirmation screen 331 (see FIG. 6) and the like.

The RAM 34 forms a work area for temporarily storing various programs read out from the storing unit 35, the various programs being executable by the CPU 31, input data, output data, parameters and the like.

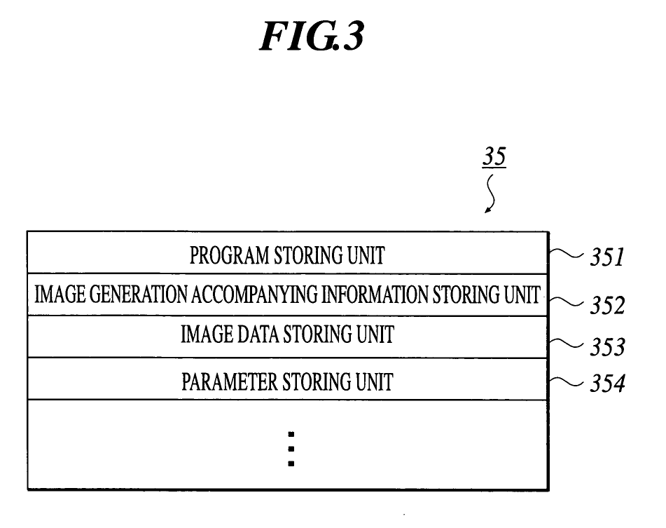

The storing unit 35 comprises an HDD (Hard Disc Drive), a nonvolatile semiconductor memory or the like. As shown in FIG. 3, the storing unit 35 comprises the program storing unit 351, an image generation accompanying information storing unit 352, an image data storing unit 353 and a parameter storing unit 354.

The program storing unit 351 stores the system program executed by the CPU 31, various processing program corresponding to the system program, such as the mamma image generation processing program, various image processing programs and the like, various application programs, etc. These programs are stored as readable program codes, and the CPU 31 executes an operation according to the program codes.

The image generation accompanying information storing unit 352 stores the inputted image generation order information and a cassette ID for identifying a cassette 17, the cassette ID being given to the cassette 17 to be used in the image generation based on the image generation order information, so as to relate each other. Here, the image generation order information is image generation reservation information of a medical image, and includes at least patient information and image generation condition (image generation part, image generation direction, distinction between left and right if the image generation part is a mamma) information.

The image data storing unit 353 stores the medical image data inputted from the reader 2 so as to relate the medical image data with the image generation accompanying information according to the cassette ID.

The parameter storing unit 354 stores an image processing parameter (a look up table in which a gradation curve to be used in a gradation process is defined, an emphasizing degree of a frequency process, and the like) so as to relate the image processing parameter with the image generation part information.

The communication control unit 36 comprises a LAN adapter, a TA (Terminal Adapter) or the like, and controls a communication between the communication control unit 36 and each apparatus connected to the network N.

The barcode reader 37 reads a cassette ID indicated by the barcode label attached on the cassette 17 to be outputted to the CPU 31.

The printer 4 is, for example, a printer taking a thermal developing system. The printer 4 output a hardcopy on which a medical image is reproduced as a visible image on a recording medium (here, a film is used) based on image-processed medical image data outputted from the controller 3 or the image server 5.

The image server 5 comprises the image DB 5a as a storing unit. The image server 5 relates the image-processed medical image data outputted from the controller 3 in the image DB 5a, so as to relate the medical image data with the image generation accompanying information.

Next, an operation in the present embodiment will be described.

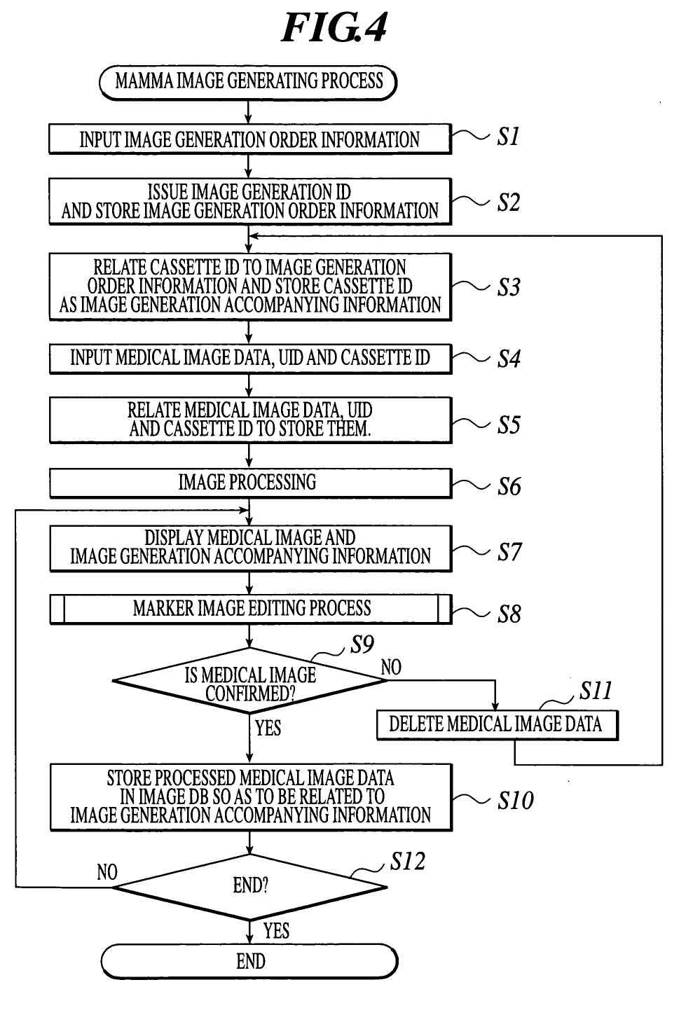

FIG. 4 shows a mamma image generating process executed by the CPU of the controller 3. The present process is realized upon generating a mamma image by a software process in combination of the CPU 31 and a mamma image generating processing program stored in the program storing unit 31.

First, a screen for inputting image generation order information (not shown) is displayed on the display unit 33. When image generation order information such as patient information, an image generation condition and the like is inputted and an input confirmation in instructed (Step S1), identification information for identifying each of the inputted image generation order information (here, image generation ID) is issued and stored in the image generation accompanying information storing unit 352 so as to relate the image generation ID with the inputted image generation order information (Step S2).

Since a diagnosis of a mamma is performed by an interpretation comparing related images, between left and right or in different directions, in general, with respect to one subject, a plurality of times of image generations under different image generation conditions are performed such as a left mamma in up and down directions (CC-L), a left mamma in an oblique direction (MLO-L), a right mamma in up and down directions (CC-R) and a right mamma in an oblique direction (MLO-R). Here, since image generation order information is inputted at each time of one image generation unit, when a mamma image generation is performed, a plurality of pieces of image generation order information are inputted at a time with respect to one subject. In the present embodiment, when all the image generation order information with respect to one subject are inputted at a time in Step S1, it is assumed that an input confirmation is instructed. In addition, when an RIS/HIS or the like is connected to the system through the network N, the image generation order information may be inputted by receiving the image generation order information from the RIS/HIS or the like.

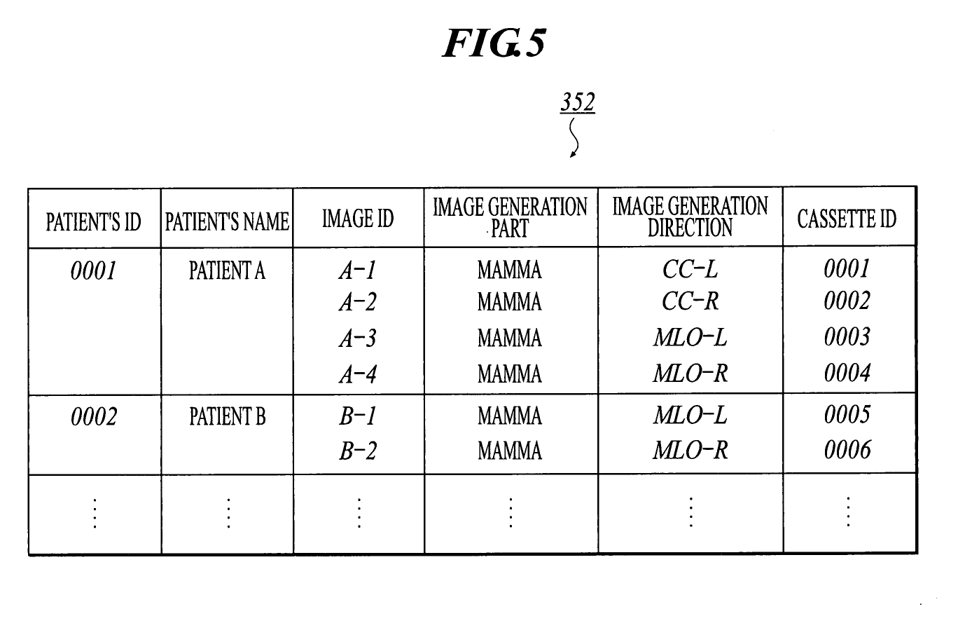

The inputted plurality of pieces of image generation order information are displayed on the display unit 33, and certain image generation order information is selected among the displayed plurality of pieces of image generation order information through the operating unit 32. Then, when a barcode shown on the cassette to be used for the image generation is read by the barcode reader 37 to input the cassette ID, the selected image generation order information is related to the cassette ID to be stored in the image generation accompanying information storing unit 352 as image generation accompanying information (Step S3). All the image generation order information with respect to one subject, which is inputted in Step S2, is sequentially related to the cassette ID. FIG. 5 shows a data storing example in the image generation accompanying information storing unit 352.

After the image generation order information is related to the cassette ID and stored as image generation accompanying information, in the image generating apparatus 1, based on the image generation condition and the cassette ID included in the image generation accompanying information stored in Step S3, a subject M (here, mamma) is radiographed by using the cassette 17 having the cassette ID included in the image generation accompanying information, and radiation image information based on radiation dose transmitted through the subject M is recorded in the photostimulable phosphor sheet 18 of the cassette 17 as a latent image. Upon the image generation, in order to avoid a mistake between a left or right mamma image or a mistake on an image generation direction, the image generation is performed with a lead marker for giving information at the image generation such as a distinction of a left or right mamma (L, R), an image generation direction (MLO, CC) and the like placed along with the subject M, and images of the subject and the marker are together recorded on the photostimulable phosphor sheet 18.

When all the cassettes 17 to which the image generation is completed are inserted to the reader 2 one by one in the turn of the image generation, the barcode shown on the inserted cassette 17 is read and the radiation image recorded in the cassette 17 is read. To medical image data (here, mamma image data) read by the reader 2, UID (Unique ID) which is a time base ID or the like is given and sequentially transmitted along with the cassette ID to the controller 3 through the communication control unit 36.

Further, upon generating a mamma image, a plurality of image generations with respect to one subject are used to performed by one image generation technician. There is a case taking a flow in which the technician inserts the cassette 17 to which the image generation is completed to the reader 2 at each time of completing one image generation, and while this operation, the image generating apparatus 1 is rotated and position-controlled for the next image generation (for example, oblique (image generation position of MLO)→horizontal (image generation position of CC)) and a cassette 17 for the next image generation is set to the image generating apparatus 1. In any case, the time-base UID, which is given to the medical image data read by the reader 2 becomes information indicating the turn of the image generation.

In addition, a method for identifying the turn of the image generation is not limited to the above-mentioned method, and, needless to say, a different method is applicable.

When the medical image data, the UID and the cassette ID are inputted from the reader 2 through the communication control unit 36 (Step S4), the received medical image data is related with the UID and the cassette ID to be stored in the image data storing unit 353 (Step S5). According to the cassette ID related to the medical image data and the cassette ID included in the image generation accompanying information, the medical image data is related to the image generation accompanying information. Then, each image processing program stored in the program storing unit 351 of the storing unit 35 is read out and an image processing is sequentially applied to the medical image data (Step S6).

The image processing is, for example, a process to manipulate inputted medical image data into appropriate one for a diagnosis, such as a gradation process, a frequency processing, format processing and the like. An image processing parameter corresponding to image generation part information included in the image generation accompanying information of the medical image data is read out from the parameter storing unit 354, and the image processing is performed based on the read image processing parameter.

Further, a density correcting process for analyzing the medical image data, for recognizing a subject area and for converting a density of an area other than the subject area into a predetermined density, for example, a value having a higher density than a minimum density value of the subject area, is performed. Here, as lead has an extremely high X-ray absorbability and therefore does not let an X-ray transmit through, image information obtained by radiographing a lead marker (marker image) has an extremely low density value compared to a density of the area other than the subject area. Accordingly, with a predetermined density threshold provided, when a density correction is performed, in order to keep the marker image, the density correction is performed on an area having a density of not less than the density threshold within the area other than the subject area. Thereby, a density of the area other than the subject area on the medical image and a density of an area other than the marker image area are corrected to be uniform.

The identification of the subject area is, for example, performed by analyzing the medical image data and extracting a contour of the subject, or the like. For example, density data of the medical image data is binarized by using a certain threshold, and a contour is defined by tracing the borderline between “0” and “1”, and thereby a subject area is determined according to the borderline, the image generation part and the image generation direction. Alternatively, a subject area may be determined by using a contour extracting method of an area corresponding to a predetermined anatomical structure of a human body area or within a human body (Journal of Japan Association of Breast Cancer Screening, Vol.17,No.1,pp87-102,1998, JP-Tokukaisyo-63-240832A).

Then, the image confirmation screen 331 is displayed on the display screen of the display unit 33, and a medical image based on the image-processed medical image and the image generation accompanying information corresponding to the medical image are simultaneously displayed (Step S7). Then, a marker image editing process is performed (Step S8). By the above-mentioned Step S7, a control section of claim 10 of the present invention is realized. Further, by Steps S7 and S8, a marker image editing section is realized.

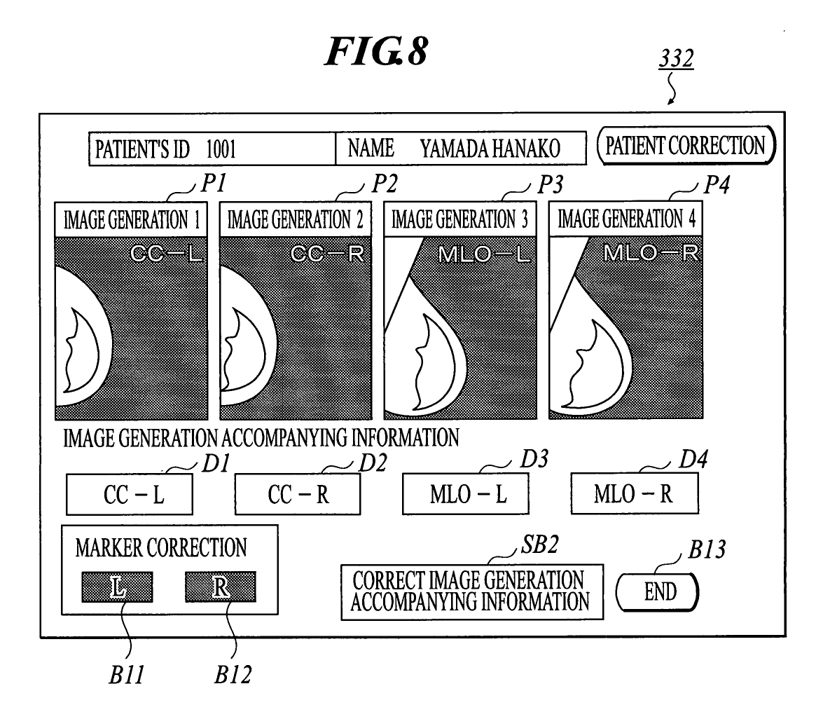

FIG. 6 shows one example of the image confirmation screen 331. As shown in FIG. 6, an image display field 331a for displaying a medical image which was read by the reader 2 and to which the image processing was applied, a patient information display field 331b for displaying patient information among the image generation accompanying information corresponding to the medical image, an image generation condition information display field 331c for displaying image generation condition information indicating an image generation direction and a distinction between a left and right mamma among the image generation accompanying information corresponding to the medical image, buttons B1 to B7 for editing the marker image on the medical image, a button B8 for displaying an image generation turn display screen 332 (see FIG. 8) for displaying a plurality of medical images generated with respect to one subject in the turn of the image generation, a confirming button B9 for instructing a confirmation of the displayed medical image, an NG button B10 for instructing a cancellation of data of the displayed medical image, and an image generation accompanying information correcting button SB1 for correcting image generation accompanying information, are provided on the image confirmation screen 331.

FIG. 6 shows an example in which a medical image on which a subject image O and a marker image A structured by a combination of a marker image indicating an image generation direction (MLO) and a marker image indicating a distinction of left or right (R) are displayed. When a lead marker indicating only an image generation direction is radiographed along with the subject M upon generating an image, only a marker image indicating the image generation direction is formed on the medical image. When a lead marker indicating only a distinction of left or right is radiographed, a marker image indicating only the distinction of left or right is formed on the medical image.

Among the buttons B1 to B7, a button B1 is a button for adding a marker image indicating “MLO” on the medical image, a button B2 is a button for adding a maker image indicating “CC” on the medical image, a button B3 is a button for adding a maker image indicating “L” on the medical image, a button B4 is a button for adding a maker image indicating “R” on the medical image, a button B5 is a button for correcting the marker image indicating “CC” to the marker image indicating “MLO”, a button B6 is a button for correcting the marker image indicating “MLO” to the marker image indicating “CC”, and a button B7 is a button for deleting a marker image within an assigned area.

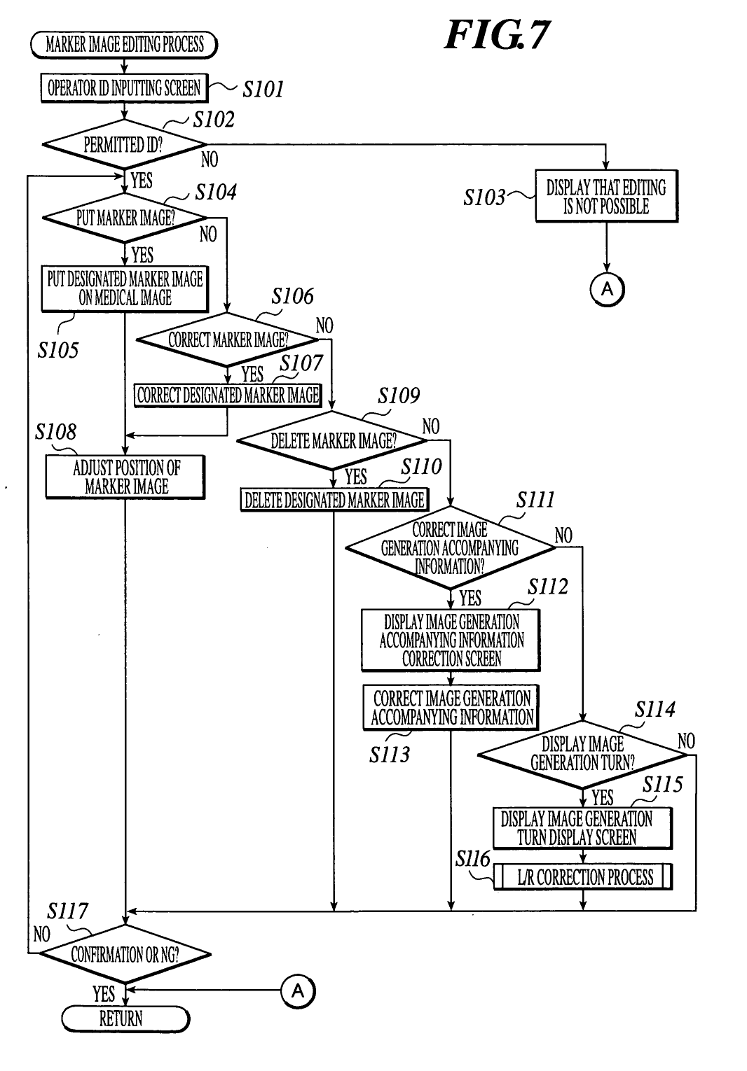

FIG. 7 shows a marker image editing process executed by the CPU 31 in Step S7 of FIG. 4. The marker image editing process is realized by a software process in combination of the CPU 31 and the marker image editing processing program stored in the storing unit 35.

First, an operator ID input screen (not shown) as an operator identification information inputting section for inputting operator identification information (here, operator ID) is displayed to be overlaid on the image confirmation screen 331 displayed on the display unit 33 (Step S101). When an operator ID is inputted from the operator ID input screen and the inputted operator ID is a permitted ID (Step S102; YES), the overlay display is cancelled and the process goes to Step S104. When the inputted operator ID is not a permitted ID (Step S102; NO), information indicating that editing is not possible is displayed on the display unit 33 (Step S103), and the process goes to Step S9 of FIG. 4.

In Step S104, an area to which an operator desires to put a marker image on the medical image displayed on the image display field 331a of the image confirmation screen 331 is designated by the operation unit 32, and when adding of the marker image is instructed by pushing one of the buttons B1 to B4 (Step S104; YES), the marker image corresponding to the pushed button is added on the designated area on the medical image (Step S105). By the above-mentioned Steps S104 to S105, it is possible to add a marker image on the medical image.

A marker image area to be corrected is designated on the medical image displayed on the image display field 331a of the image confirmation screen 331 by the operation unit 32, and correcting of the marker image is instructed by designating a letter of a marker image after the correction according to pushing one of the buttons B5 and B6 (Step S106; YES). Then, after a density of the marker image existing in the designated area on the medical image is converted to a density equal to a density of an area other than the subject area, the marker image corresponding to the pushed button is added on the designated area on the medical image, whereby the marker image is corrected (Step S107). By the above-mentioned Steps S106 to S107, a correcting section of claim 1 of the present invention is realized, and it is possible to correct a marker image (marker image indicating an image generation direction (MLO or CC)) on the medical image.

When a position adjustment of the marker image that is added or corrected by operating the operation unit 32 such as a mouse or the like is inputted, a position of the marker image is adjusted according to the input (Step S108).

When a marker area that an operator desires to delete from the medical image displayed on the image display field 331a of the image confirmation screen 331 is designated and a deletion of the marker image is instructed by pushing the button B7 (Step S109; YES), by converting a density of the marker image existing in the designated area on the medical image into a density equal to an area other than the subject area, the marker image is deleted (Step S110). By the above-mentioned Steps S109 to S110, it is possible to delete a marker image on the medical image.

When the button Sb1 is pushed by the operating unit 32 for instructing a correction of image generation accompanying information (Step S111; YES), image generation accompanying information corresponding to the displayed medical image is read out from the image generation accompanying information storing unit 352, and an image generation accompanying information correction screen (not shown) in which the read image generation accompanying information is shown is displayed (Step S112). When an input for correcting the image generation accompanying information is made on the image generation accompanying information correction screen by the operating unit 32 (an input for correcting a cassette ID is made), information of the image generation accompanying information storing unit 352 is corrected based on the inputted information (Step S113), and the process goes to Step S117. By the above-mentioned Steps S111 to S113, a correcting section of claim 10 of the present invention is realized. With a relation between image generation order information and a cassette ID changed by correcting a cassette ID included in image generation accompanying information, it is possible to correct a relation between the image generation accompanying information and the medical image.

On the other hand, when the button B8 is pushed by the operating unit 32 and a display of the image generation turn display screen 332 is instructed (Step S114; YES), the image generation turn display screen 332 is displayed on the display screen of the display unit 33 (Step S115). Since an UID indicating an image generation turn is given to each medical image data, it is possible to easily rearrange the medical images in the image generation turn. When the image generation turn display screen 332 is displayed, an L/R correction process (described later) is executed (Step S116). By the above-mentioned Step S115, a control section of claims 13 and 14 of the present invention is realized, and by Step S116, a correcting section of claims 13 and 14 of the present invention is realized. Thereby, it is possible to do the correction when a distinction of left or right (L, R) mamma of image generation accompanying information and that of a marker image on the medical image are different.

In other words, simply correcting of L/R of an individual image, which may result in a mistake on a part, is prohibited, and correcting of L/R is only permitted after the judgment using the above-mentioned flow in which a relation with the related images is considered. Further, when all the related images with respect to one subject are displayed and if the number of marker image L and the number of marker image R given to each image are not the same (supposed to be 2:2), the display may be switched to the above-mentioned image generation turn display screen 332 for making it possible for an operator to identify which medical image has a wrong marker image. In this case also, it is preferable to display image generation accompanying information of each image along with each image. However, it is not necessary for a judgment of which image's marker of L/R is wrong among the related images.

As a result of displaying the image confirming screen 331, when image generation directions are different between one indicated by the image generation accompanying information and one indicated by the marker image, such as the case that an image generation direction included in the image generation accompanying information is MLO and an image generation direction indicated by the marker image is CC, it is possible for an operator such as an image generation technician or the like to identify a correct image generation direction from a displayed subject image. When the marker image does not indicates the correct image generation direction identified from the subject image, by correcting MLO/CC of the marker image, it is possible to make image generation directions correspond each other between the marker image and the image generation accompanying image based on the correct information. When the marker image indicates the correct image generation direction identified from the subject image, the cassette ID included in the image generation accompanying information is corrected and a relation between the image generation order information and the cassette ID is changed for correcting the relation between the image generation accompanying information and the medical image. Thereby, it is possible to make image generation directions correspond each other between the marker image and the image generation accompanying image based on the correct information.

On the other hand, when there is a difference on the distinction of left or right (L, R) between the image generation accompanying information and the marker image, it is difficult for an operator such as an image generation technician or the like to identify correct information by viewing the subject image. Accordingly, the image generation turn display screen 332 (see FIG. 8) is displayed according to a push of the button B8 in Step S114 by an operator such as an image generation technician or the like and medical images are displayed in the image generation turn. Thereby it is possible for the operator to identify the correct-information.

FIG. 8 shows one example of the image generation turn display screen 332. As shown in FIG. 8, on the image generation turn display screen 332, image display fields P1 to PN for displaying N (here, four) pieces of medical images generated with respect to one subject in the turn of the image generation, and image generation accompanying information display fields D1 to DN for displaying an image generation condition indicating an image generation direction and a distinction of a left or right mamma included in the image generation accompanying information corresponding to each of the displayed medical images are provided. Here, the image generation accompanying information corresponding to each of the displayed medical images is the image generation accompanying information related with each medical image data in Step S5 according to the cassette ID. In other words, by displaying the image generation turn display screen 332, a plurality of medical images including a marker image that are generated with respect to one subject are simultaneously displayed in the turn of the image generation so as to relate each medical image with the corresponding image generation accompanying information.

Further, below the screen, for correcting the medical image, a button B11 for correcting a marker image indicating “R” on the medical image displayed on the image confirmation screen 331 to a marker image indicating “L”, a button B12 for correcting the marker image indicating “L” on the medical image to the marker image indicating “R”, an image generation accompanying information correcting button SB2 for correcting the image generation accompanying information, and an “END” button B13 for ending the L/R correction process and returning to the image confirmation screen 331, are provided.

Normally, upon generating a mamma image, due to a pain caused by a pressure plate, generating an image of a mamma of one side is not continuously performed. In other words, when four pieces of mamma images are to be generated, upon reserving image generations, for example, cassette IDs of cassettes to be used are related with each image generation order information in Step S3 so as to make the turn of the image generation as (1) MLO-L→(2) MLO-R→(3) CC-L→(4) CC-R. On the other hand, when each of the above-mentioned cassettes is read by the reader 2 and the read medical images are displayed, if a marker image of a medical image which was first read and displayed is MLO-R, all the medical images with respect to the same subject are displayed in the turn of the image generation by pushing the image generation turn display button B8, and if the marker images on the medical images follow: (1) MLO-R→(2) MLO-L→(3) CC-R→(4) CC-L, it means that the marker images formed on each medical image indicate distinctions of left or right (L/R) of the subject correctly. In this case, in the L/R correction process (described later), by correcting the cassette ID for changing a relation between the cassette ID (see FIG. 5) included in the image generation accompanying information and the image generation order information, the relation between the image generation accompanying information and the medical image is corrected, whereby it is possible to make the marker image correspond to information indicating a distinction of left or right in the image generation accompanying information.

On the contrary, as a result of arranging the medical images in the turn of the image generation, if there is a medical image having a marker image's distinction of left or right which does not follow L→R→L→R or R→L→R→L, it means that the marker image formed on the medical image does not correctly indicate information of the subject image. For example, when the marker images on the medical images arranged in the turn of the image generation are (1) L→(2) R→(3) R→(4) R, and the corresponding image generation accompanying information is (1) L→(2) R→(3) L→(4) R, it means that an image generation of (3) was performed with a wrong marker placed. Accordingly, in the L/R correction process (described later), by correcting the marker image of (3) to L, it is possible to make the marker image correspond to information indicating a distinction of left or right in the image generation accompanying information.

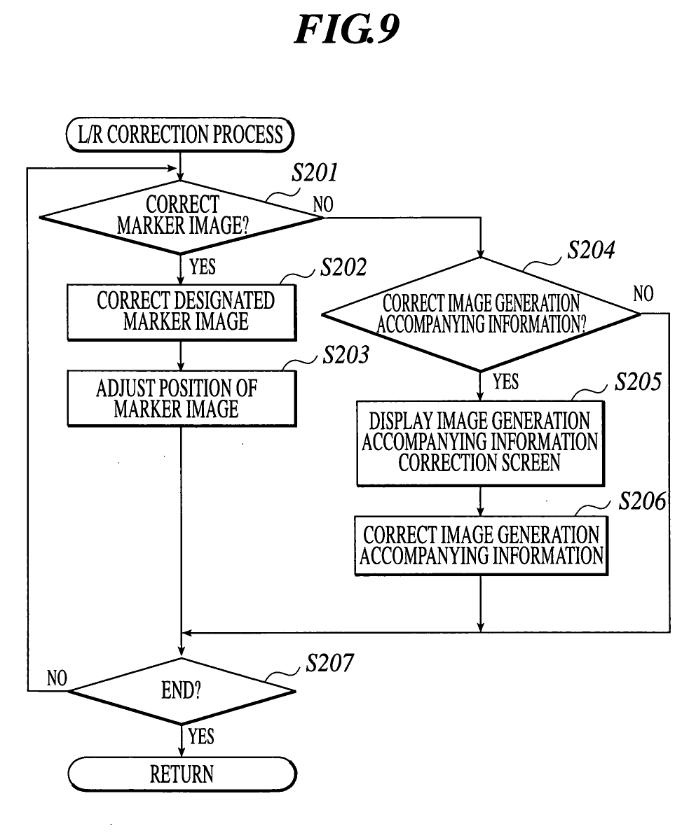

FIG. 9 shows the L/R correction process executed by the CPU 31 in Step S116 of FIG. 7. The L/R correction process is realized by a software process in combination of the CPU 31 and the L/R correction processing program stored in the storing unit 35.

When an L or R marker image area to be corrected is designated on the medical image (on the medical image displayed on the image display field 331a of the image confirmation screen 331) displayed on the image generation turn display screen 332 for the correction, and correction of the marker image is instructed by designating a letter after the correction by pushing one of the buttons B11 and B12 (Step S201; YES), after a density of the marker image existing within the designated area on the medical image is converted to a density equal to an area other than the subject area, a marker image corresponding to the pushed button is added on the designated area on the medical image, whereby the marker image is corrected (Step S202). When a position adjustment of the corrected marker image is inputted by an operation of a mouse or the like of the operating unit 32, a position of the marker image is adjusted according to the input (Step S203), and the process goes to Step S207.

On the other hand, if a correction of a marker image is not instructed by the operating unit 32 (Step S201; NO), but a correction of image generation accompanying information is instructed by pushing the button SB2 (Step S204; YES), image generation accompanying information corresponding to the displayed plurality of medical images is read out from the image generation accompanying information storing unit 352, and the image generation accompanying information correction screen (not shown) on which the read image generation accompanying information is shown is displayed (Step S205). When an input is made for correcting the image generation accompanying information on the image generation accompanying information correction screen by the operating unit 32, information of the image generation accompanying information storing unit 352 is corrected based on the inputted information (Step S206), and the process goes to Step S207.

When the processes of Steps S201 to S206 are completed, and the completion is instructed by pushing the button B13 at the operating unit 32 (Step S207; YES), the image confirmation screen 331 is re-displayed on the display unit 33, and the process goes to Step S117 of FIG. 7.

In FIG. 7, until a confirmation of the displayed medical image is instructed by pushing the confirming button B9 from the image confirmation screen 331, or until a cancellation of the displayed medical image is instructed by pushing the NG button B10, processes of Steps S104 to S116 are executed based on an operation signal of the operation unit 32. When the confirming button B9 or the NG button B10 is pushed and the confirmation or the cancellation of the displayed medical image is instructed (Step S117; YES), the process goes to Step S9 of FIG. 4.

In Step S9 of FIG. 4, when an instruction from the operation unit 32 is the confirmation of the medical image (Step S9; YES), in Step S10 as a relating section, the processed medical image data is related with the image generation accompanying information to be transmitted to the image server 5 through the communication control unit 36 and stored in the image DB 5a so as to relate the medical image data with the image generation accompanying information (Step S10). When an instruction from the operation unit 32 is not the confirmation of the medical image, that is, when an instruction is the cancellation of the medical image (Step S9; NO), the medical image data stored in the image data storing unit 353 is deleted (Step S11). When Steps S7 to S11 are executed to all the medical images generated under a plurality of image generation conditions with respect to one subject and completed (Step S12; YES), the present process ends.

By reading out and transmit the medical image data stored in the image DB 5a to the printer 4, it is possible to record the medical image on which a marker image is correctly shown on-the film and to output it. In addition, the medical image may be recorded on the film and outputted by directly transmitting the medical image data from the controller 3 to the printer 4.

As described above, according to the controller 3, it is possible to edit a marker image of a lead marker indicating a distinction of left or right mamma and indicating an image generation direction. Therefore, when an image generation is performed without a marker or when there is a mistake on a marker, it is possible to prevent a medical image from including a wrong marker image to be stored or outputted.

Further, since a marker image included in a medical image and image generation accompanying information corresponding to the medical image are simultaneously displayed on the display screen of the display unit 33, it is possible for an operator such as an image generation technician or the like to compare between the marker image included in the medical image and the image generation accompanying information corresponding to the medical image to see if there is a difference, and if there is a difference, it is possible to correct the marker image included in the displayed medical image or to correct the image generation accompanying information corresponding to the medical image. Furthermore, since it is possible to display a plurality of medical images (mamma images) generated with respect to the same subject in the turn of the image generation, after an operator such as an image generation technician or the like identifies whether the marker image on the medical image correctly indicates information upon the image generation, it is possible to correct the marker image on the medical image or to correct the image generation accompanying information corresponding to the medical image.

In addition, described contents in the above-mentioned embodiment are a suitable example of the medical image generating system 100 relating to the present invention, and the present invention is not limited to the contents.

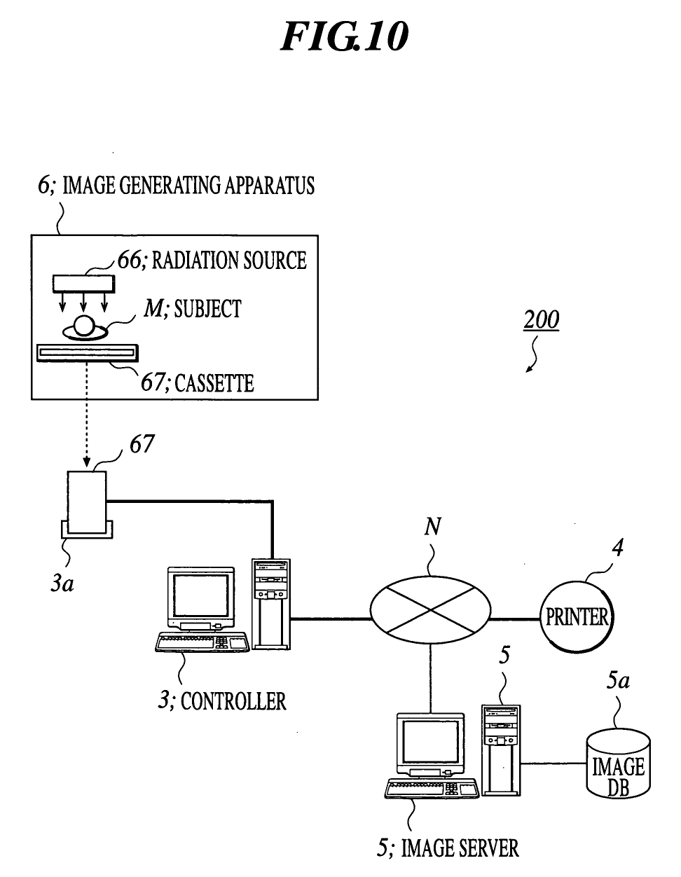

For example, in the above-mentioned embodiment, the case that the present invention is applied to the medical image generating system 100 comprising a CR apparatus using a cassette is described. However, the present invention is not limited to such a case. For example, it is possible to apply the present invention to a medical image generating system 200 using a radiation detector such as an FPD (flat panel detector) shown in FIG. 10, etc.

As shown in FIG. 10, the medical image generating system 200 comprises an image generating apparatus 6, the controller 3, the printer 4, the image server 5, etc. In the system, the controller 3, the printer 4 and the image server 5 are connected through the network N so as to make it possible to mutually transmit/receive data. Further, the controller 3 comprises an I/F to be connected with a cradle 3a, in addition to the structure shown in FIG. 2. By attaching a cassette 67 to the cradle 3a, the controller 3 is connected to the cassette 67 so as to make it possible to mutually transmit/receive data. Here, the number of each apparatus is not specifically limited. Further, an external apparatus or a system such as a viewer, an HIS/RIS or the like may be connected to the network N.

The image generating apparatus 6 comprises a radiation source 66 for irradiating a radiation to a subject M to perform an image generation. The cassette 67 is a cassette type image generating apparatus in which an FPD for detecting a radiation image transmitted through the subject is installed. Medical image data is obtained by converting the detected radiation image to an electrical signal. On the cassette 67, a barcode indicating a cassette ID for identifying the cassette is shown.

In the above-mentioned medical image generating system 200, the controller 3 performs the mamma image generating process shown in FIG. 4. Then, when the medical data and the cassette ID of the cassette 67 is transformed from the cassette 67 through the cradle 3a, the medical image data is related with image generation accompanying information according to the cassette ID, and stored in the image data storing unit 353. Then, an image processing and a marker editing process are applied to the medical image data, and the processed medical image data is related with the image generation accompanying information to be stored in the image DB 5a of the image server.

When a mamma image is generated by using an FPD, unlike a case of the system using the above-mentioned CR apparatus where a plurality of cassettes are used, a plurality of image generations are performed with one cassette by repeating: generating an image transmitting medical image data to the controller 3 delete the medical image data from the cassette 17. Accordingly, the turn of receiving data at the controller 3 side becomes the turn of the image generation, and thereby identifying the turn of the image generation upon displaying the image generation turn display screen 332 at the controller 3 side becomes easy, whereby it is more preferable. When an FPD is used, upon transmitting medical image data from the FPD, the medical image data may be transmitted to the controller 3 after a time clock of data transmittance is added to header information of the medical image data at the FPD side.

Further, the present invention is not limited to ones using the above-mentioned cassette or FPD. It is possible to apply the present invention to any medical image generating system such as a system dedicated to erect/supine type or the like.

Further, in the above-mentioned embodiment, the case that the marker editing is performed by displaying each one of mamma images as a medical image is described. Like the time of a diagnosis, two mamma images which are left and right mammas may be displayed so as to make breast-wall sides face thereof each other, while image generation accompanying information of each mamma image is displayed along with the mamma image, for performing the marker editing. Further, with medical images with respect to one subject displayed on one screen in the turn of the image generation, the marker editing and/or the image generation accompanying information correcting may be done to each medical image.

Further, in the above-mentioned embodiment, the case that a mamma image generation is performed with a lead marker placed and the marker image put on the mamma image is edited is described. It is also possible to apply the present invention to the case that an image of another part is generated with a marker placed and the marker image formed on the medical image is edited.

And so forth, detailed structures and detailed operations of each apparatus structuring the medical image generating system 100 or the medical image generating system 200 may be changed accordingly without departing the gist of the present invention.

The entire disclosure of a Japanese Patent Application No. 2004-197979, filed on Jul. 5, 2004, and a Japanese Patent Application No. 2004-198104, filed on Jul. 5, 2004, including specifications, claims, drawings and summaries are incorporated herein by reference in their entirety.

What is claimed is:

1. A medical image generating system for generating a medical image including a subject image and a marker image indicating information of an image generation by generating an image of a subject and by generating an image of a marker for providing the information of the image generation, the medical image generating system comprising:
a marker image editing section for editing the marker image.

2. The system of claim 1, wherein the marker image editing section corrects a part of or all of the marker image.

3. The system of claim 1, wherein the marker image editing section deletes a part of or all of the marker image by converting a density of the part of or all of the marker image into a density equal to a density of an area which is outside of an area of the subject image and outside of an area of the marker image, on the medical image.

4. The system of claim 1, wherein the marker image editing section adds a new marker image on an area which is outside of the subject area of the medical image when the medical image does not include the marker image.

5. The system of claim 1, further comprising a storing section for storing data of the medical image on which the marker image has been edited.

6. The system of claim 1, further comprising a relating section for relating the data of the edited medical image with image generation accompanying information relating to the medical image.

7. The system of claim 1, further comprising:
a reading apparatus for performing the image generation by using a cassette comprising a photostimulable phosphor plate and for obtaining the medical image by reading an image recorded in the photostimulable phosphor plate of the cassette; and
a control apparatus for applying a predetermined process on the medical image obtained by the reading apparatus,
wherein the control apparatus comprises the marker image editing section.

8. The system of claim 1, wherein
the medical image generating system performs the image generation by using a cassette type flat panel detector,
the medical image generating system further comprises a control apparatus for applying a predetermined process on the medical image obtained by the flat panel detector, and
the control apparatus comprises the marker image editing section.

9. The system of claim 1, wherein
the marker image editing section comprises an operator identification information inputting section for inputting operator identification information for identifying an operator, and
when the inputted operator identification information is permitted operator identification information, the editing is allowed.

10. A medical image generating system for generating a medical image including a marker image indicating information of an image generation by generating an image of a subject and by generating an image of a marker, the medical image generating system comprising:
a display section for displaying a display screen;
a control section for simultaneously displaying the medical image including the generated marker image and image generation accompanying information corresponding to the medical image; and
a correcting section for correcting the marker image included in the displayed medical image or for correcting the image generation accompanying information.

11. The system of claim 10, wherein
the medical image generating system performs the image generation by using a cassette comprising a photostimulable phosphor plate,
the medical image generating system comprises a reading apparatus for obtaining the medical image by reading an image recorded in the photostimulable phosphor plate of the cassette and an information processing apparatus for applying a predetermined process on the medical image obtained by the reading apparatus, and
the information processing apparatus comprises the display section, the control section and the correcting section.

12. The system of claim 10, wherein
the medical image generating system performs the image generation by using a cassette type flat panel detector,
the medical image generating system comprises an information processing apparatus for applying a predetermined process on the medical image obtained by the flat panel detector, and
the information processing apparatus comprises the display section, the control section and the correcting section.

13. A medical image generating system for generating a plurality of medical images with respect to one subject, the plurality of medical images including a marker image indicating information of an image generation, by performing a mamma image generation under a plurality of image generation conditions with respect to the one subject and by generating an image of a mamma and an image of a marker for providing the information of the image generation, the medical image generating system comprising:
a display section for displaying a display screen;
a control section for simultaneously displaying the plurality of medical images including the marker image generated with respect to the one subject on the display screen of the display section so as to arrange the plurality of medical images in a turn of the image generation; and
a correcting section for correcting the marker image included in the plurality of displayed medical images.

14. The system of claim 13, wherein
the control section simultaneously displays the plurality of medical images including the marker image generated with respect to the one subject on the display screen of the display section, so as to relate the plurality of medical images with image generation accompanying information corresponding to each of the plurality of medical images and to arrange the plurality of medical images in the turn of the image generation, and
the correcting section corrects the marker image included in the displayed medical images or corrects the image generation accompanying information.

15. The system of claim 14, wherein
the medical image generating system performs the image generation by using a cassette comprising a photostimulable phosphor plate,
the medical image generating system comprises a reading apparatus for obtaining a medical image recorded in the photostimulable phosphor plate of the cassette and an information processing apparatus for applying a predetermined process on the medical image obtained by the reading apparatus, and
the information processing apparatus comprises the display section, the control section and the correcting section.

16. The system of claim 14, wherein
the medical image generating system performs the image generation by using a cassette type flat panel detector,
the medical image generating system comprises an information processing apparatus for applying a predetermined process on a medical image obtained by the flat panel detector, and
the information processing apparatus comprises the display section, the control section and the correcting section.

17. A medical image generating method for generating a plurality of medical images with respect to one subject, the plurality of medical images including a marker image indicating information of an image generation, by performing a mamma image generation under a plurality of image generation conditions with respect to the one subject and by generating an image of a mamma and an image of a marker for providing the information of the image generation, wherein
by simultaneously displaying the plurality of medical images including the marker image generated with respect to the one subject on the display screen of the display section in a turn of the image generation, it is allowed to correct the marker image.

18. The method of claim 17, wherein
by simultaneously displaying the plurality of medical images including the marker image generated with respect to the one subject on the display screen of the display section in a turn of the image generation, so as to relate each of the plurality of medical images with image generation accompanying information corresponding to the each of the plurality of medical images, it is allowed to correct the marker image or the image generation accompanying information.
